(19) United States
(12) Patent Application Publication
(10) Pub. No.: US 2012/0302813 A1
(43) Pub. Date: Nov. 29, 2012

(54) PROCESSES AND APPARATUSES FOR PRODUCING A SUBSTANTIALLY LINEAR PARAFFIN PRODUCT

(75) Inventors: Rajeswar Gattupalli, Arlington Heights, IL (US); Stephen W. Sohn, Arlington Heights, IL (US); Michael J. Cleveland, Northbrook, IL (US)

(73) Assignee: UOP LLC, Des Plaines, IL (US)

(21) Appl. No.: 13/117,930

(22) Filed: May 27, 2011

(51) Int. Cl. C07C 7/12; B01D 11/04; C07C 7/13

(52) U.S. Cl. 585/821; 585/820; 422/256

(57) ABSTRACT

Processes and apparatuses are provided for producing a normal paraffin product or a substantially linear paraffin product from a feed including normal hydrocarbons, non-normal hydrocarbons, and contaminants. The contaminants are extracted from the feed through contact with an ionic liquid stream to form a clean stream of hydrocarbons. The desired hydrocarbons are selectively adsorbed from the clean stream of hydrocarbons with a molecular sieve to remove the desired hydrocarbons from the non-desired hydrocarbons. A desorbent recovers the desired hydrocarbons from the molecular sieve. Then the desired hydrocarbons are separated from the desorbent to yield the normal paraffin product.

### FIELD OF THE INVENTION

The present invention relates generally to processes and apparatuses for producing a substantially linear paraffin product from a feed, and more particularly relates to processes and apparatuses for decontaminating a kerosene feed with an ionic liquid during production of a substantially linear paraffin product.

### BACKGROUND OF THE INVENTION

Kerosene is commonly used as a feedstock for the production of normal paraffins. Untreated kerosene feedstock includes normal and non-normal hydrocarbons as well as contaminants such as heteroatoms that contain nitrogen, sulfur, and oxygenates. Therefore, separation of normal and non-normal hydrocarbons is required. During this process of separation, an adsorbent unit and molecular sieve may be used. However, the presence of contaminants in the adsorbent unit and on the molecular sieve disrupts the separation process. In fact, the presence of contaminants in the adsorbent unit may render the entire normal paraffin production process uneconomical.

Currently, to avoid contamination of the adsorbent unit, the kerosene is pretreated to eliminate the contaminants. During a typical pretreatment process, the kerosene feed is hydrotreated at severe conditions to remove the heteroatoms containing nitrogen, sulfur, and oxygenates. Hydrotreating involves treatment of kerosene with hydrogen in the presence of a catalyst. For example, for sulfur contaminants, hydrotreating results in their conversion to hydrogen sulfide, which is separated and then converted to elemental sulfur. Unfortunately, this type of processing is typically quite expensive because it requires a source of hydrogen, high pressure process equipment, expensive hydrotreating catalysts, and a sulfur recovery plant for conversion of the resulting hydrogen sulfide to elemental sulfur.

Accordingly, it is desirable to provide processes and apparatuses for producing a substantially linear paraffin product from a kerosene feed without requiring hydrotreatment at severe conditions. Also, it is desirable to provide processes and apparatuses for producing a substantially linear paraffin product from a kerosene feed, wherein the processes and apparatuses utilize ionic liquids to remove contaminants from the kerosene feed. Furthermore, other desirable features and characteristics of the present invention will become apparent from the subsequent detailed description of the invention and the appended claims, when taken in conjunction with the accompanying drawing and this background of the invention.

### SUMMARY OF THE INVENTION

Processes and apparatuses are provided for producing a substantially linear paraffin product from a feed including normal hydrocarbons, lightly-branched non-normal hydrocarbons, heavier-branched non-normal hydrocarbons, and contaminants. As used herein, “substantially linear paraffin product” includes normal hydrocarbons and lightly-branched non-normal hydrocarbons. Further, as used herein, “lightly-branched non-normal hydrocarbons” include isoparaffins having no more than two methyl groups and no other branches. Also, as used herein, “heavier-branched non-normal hydrocarbons” include aromatics, isoparaffins having more than two methyl groups, and isoparaffins having at least one branch longer than a methyl group.

In accordance with one embodiment, a process for producing a substantially linear paraffin product from a feed including normal hydrocarbons, lightly-branched non-normal hydrocarbons, heavier-branched non-normal hydrocarbons and contaminants include contacting the feed with an ionic liquid stream and extracting the contaminants to form a clean stream of hydrocarbons. Then, the process provides for selectively adsorbing the normal hydrocarbons and lightly-branched non-normal hydrocarbons from the clean stream of hydrocarbons with a molecular sieve to remove the normal hydrocarbons and lightly-branched non-normal hydrocarbons from the heavier-branched non-normal hydrocarbons. Thereafter, the normal hydrocarbons and lightly-branched non-normal hydrocarbons are recovered from the molecular sieve with a desorbent. Further, the normal hydrocarbons and lightly-branched non-normal hydrocarbons are separated from the desorbent to yield the substantially linear paraffin product.

In another embodiment, a process for producing a normal paraffin product from a feed including normal hydrocarbons, non-normal hydrocarbons and contaminants is provided. In the process, the feed is contacted with an ionic liquid stream to extract contaminants from the feed into the ionic liquid stream to create a stream of clean stream of hydrocarbons and a stream of dirty ionic liquid. Further, the clean stream of hydrocarbons is fed to a molecular sieve that selectively adsorbs normal hydrocarbons from the clean stream of hydrocarbons. The normal hydrocarbons are recovered from the molecular sieve with a desorbent. Thereafter, the normal hydrocarbons are separated from the desorbent to yield the normal paraffin product.

An apparatus configured to produce a substantially linear paraffin product from a feed including normal hydrocarbons, lightly-branched non-normal hydrocarbons, heavier-branched non-normal hydrocarbons and contaminants in accordance with a further embodiment comprises an extraction column configured to contact the feed with an ionic liquid stream to extract the contaminants from the feed into the ionic liquid stream to form a clean stream of hydrocarbons and a dirty ionic liquid stream. Further the apparatus includes an adsorbent chamber configured to receive the clean stream of hydrocarbons. Also, a molecular sieve is located in the adsorbent chamber and configured to selectively adsorb the normal hydrocarbons and lightly-branched non-normal hydrocarbons from the clean stream of hydrocarbons to remove the normal hydrocarbons and lightly-branched non-normal hydrocarbons from the heavier-branched non-normal hydrocarbons to create a raffinate. Further, the apparatus includes a desorbent configured to recover the normal hydrocarbons and lightly-branched non-normal hydrocarbons from the molecular sieve to create an extract. An extract column is configured to receive the extract and separate the normal hydrocarbons and lightly-branched non-normal hydrocarbons from the desorbent to yield the substantially linear paraffin product. The apparatus also provides for a raffinate column configured to receive the raffinate and separate the heavier-branched non-normal hydrocarbons from the desorbent. In addition, a rotary valve is configured to control entry of the clean stream of hydrocarbons and the desorbent into the adsorbent chamber, and control removal of the extract and the raffinate from the adsorbent chamber. An extraction stage is configured to treat the clean stream of hydrocarbons with a solvent stream, such as water, to extract any ionic liquid from the clean stream of hydrocarbons and to produce a solvent and ionic liquid stream. Further, a separator is configured to receive the solvent and ionic liquid stream, to mix solvent with the dirty ionic liquid stream to remove the contaminants from the dirty ionic liquid, and to form a cleaned ionic liquid stream. Finally, an evaporator is configured to remove the solvent from the cleaned ionic liquid stream to form a regenerated ionic liquid stream for recycling to the ionic liquid stream and a regenerated solvent stream for recycling to the extraction stage.

### BRIEF DESCRIPTION OF THE DRAWINGS

Embodiments of the present invention will hereinafter be described in conjunction with the following drawing FIGURE, wherein:

FIG. 1 schematically illustrates an apparatus for producing a normal paraffin product in accordance with an exemplary embodiment.

### DETAILED DESCRIPTION

The following Detailed Description is merely exemplary in nature and is not intended to limit the invention or the application and uses of the invention. Furthermore, there is no intention to be bound by any theory presented in the preceding Background of the Invention or the following Detailed Description.

Processes and apparatuses for producing a normal or substantially linear paraffin product from a kerosene feed are provided herein. In accordance with an exemplary process, the kerosene feed is initially fractionated to obtain a heart cut of kerosene containing C₅ to C₁₀ hydrocarbons, C₁₀ to C₁₃ hydrocarbons, C₁₀ to C₁₈ hydrocarbons, or another range of desired hydrocarbons. (As used herein, molecules with carbon chains having X carbons will be designated C_{X}.) Specifically, light hydrocarbons (lighter than the desired hydrocarbons) and heavy hydrocarbons (heavier than the desired hydrocarbons) are removed from the feed, leaving the heart cut of kerosene.

The heart cut of kerosene is then contacted with an ionic liquid stream in a vessel such as, for example, a counter-current extraction apparatus. Due to this contact, the contaminants within the heart cut of kerosene, including, for example, heteroatoms containing nitrogen, sulfur, and oxygenates, are extracted from the kerosene.

As a result of the extraction of contaminants into the ionic liquid, a substantially clean stream of hydrocarbons and a dirty ionic liquid stream are created.

In certain embodiments, the ionic liquid stream may be used to remove a substantial amount of contaminants, but a follow up processing of the feed may be desired for removal of substantially all contaminants. Specifically, the substantially clean stream of hydrocarbons may undergo further processing at mild hydroprocessing conditions, i.e., significantly reduced hydrogen partial pressure, higher liquid hourly space velocity (LHSV), and lower temperatures.

In certain embodiments, ionic liquid may be entrained or otherwise caught in the clean stream of hydrocarbons. To remove the ionic liquid from the clean stream of hydrocarbons, the process may provide for contacting the clean stream of hydrocarbons with a solvent stream, such as water, in one or more stages. During this treatment, the ionic liquid is extracted from the clean stream of hydrocarbons into the solvent stream.

Thereafter, the normal hydrocarbons or the normal hydrocarbons and lightly-branched non-normal hydrocarbons in the clean stream of hydrocarbons are selectively adsorbed by a molecular sieve, such as a zeolite. Then, the adsorbed hydrocarbons are recovered from the molecular sieve by a desorbent. To yield the normal or substantially linear paraffin product, the recovered hydrocarbons are separated from the desorbent.

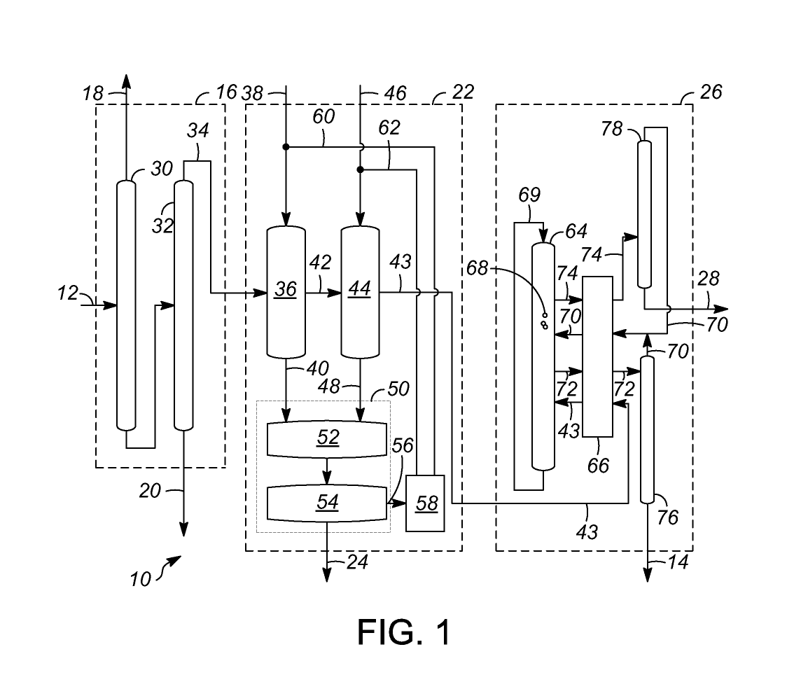

Referring to FIG. 1, an apparatus for producing a normal or substantially linear paraffin product in accordance with an exemplary embodiment is shown and generally designated 10. For purposes of the exemplary embodiment, the apparatus 10 processes a kerosene feed 12 that includes normal hydrocarbons, non-normal hydrocarbons (lightly-branched and heavier-branched) and contaminants to remove the desired paraffins to produce a desired paraffin product 14, e.g., a normal paraffin product, or a substantially linear paraffin product. As shown, the apparatus 10 can be considered to include three sections: first, a fractionation section 16 for removing light kerosene 18 and heavy kerosene 20; second, a decontamination section 22 for removing contaminants 24 such as heteroatoms containing nitrogen, sulfur, and oxygenates; and third, a separation section 26 for separating non-desired hydrocarbons 28 from the desired hydrocarbons resulting in the desired paraffin product 14.

Referring first to the fractionation section 16, it can be seen that the apparatus 10 provides a first fractionation column 30 and a second fractionation column 32. Feed 12 passes first to the first fractionation column 30 where light kerosene 18 comprising hydrocarbons with a molecular weight less than desired is removed. Feed 12 then passes to the second fractionation column 32 where heavy kerosene 20 comprising hydrocarbons with a molecular weight greater than desired is removed. As a result, the fractionation section 16 results in a heart cut of kerosene 34 containing hydrocarbons with a desired range of molecular weights that is fed to the decontamination section 22. While fractionation section 16 is illustrated with a first fractionation column 30 and a second fractionation column 32, it will be appreciated that fractionation section 12 may have more fractionation columns as is necessary to remove light kerosene 18 and heavy kerosene 20.

The heart cut of kerosene 34 passes to an extraction apparatus 36, such as, for example, a counter current extraction column, of decontamination section 22 for extraction of contaminants 24. The extraction apparatus 36 receives an ionic liquid stream 38, for example, as an overhead stream, that contacts the heart cut of kerosene 34 to cause extraction of contaminants 24 through a phase separation. Specifically, contaminants 24 within the heart cut of kerosene 34 are extracted therefrom into the ionic liquid 38 to create a phase containing dirty ionic liquid stream 40 and a phase containing the clean stream of hydrocarbons 42. The dirty ionic liquid stream 40 contains ionic liquid and the contaminants extracted from the kerosene. For purposes of the embodiment, the particular ionic fluid is selected for use based on its ability to extract the forms of nitrogen, sulfur, and oxygenates present in the kerosene feed. The ionic fluid may be, for example, imidizolium ionic liquid, phosphonium ionic liquid, or another ionic liquid.

In an exemplary embodiment, the clean stream of hydrocarbons 42 passes to an extraction stage or stages 44 for removal of any ionic liquid entrained in the clean stream of hydrocarbons 42 As shown, a solvent stream 46, such as water, is introduced into extraction stage 44. To remove any ionic liquid remaining in the clean stream 42, the clean stream 42 contacts the solvent stream 46 and, during this contact, any ionic liquid entrained in the clean stream 42 is dissolved into the solvent stream 46 to form a solvent and ionic liquid stream 48. With the removal of ionic liquid, the clean stream of hydrocarbons, now clean stream of hydrocarbons 43, is ready for further hydrocarbon processing in the separation section 26, discussed in further detail below. However, in certain embodiments, the clean stream of hydrocarbons 43 may first be processed by a hydroprocessor (not shown) at mild hydroprocessing conditions in order to remove any remaining contaminants.

In an exemplary embodiment, both the dirty ionic liquid stream 40 and the solvent and ionic liquid stream 48 pass into a separator 50. As shown, the separator 50 includes two units, an upstream stripper 52 and a downstream settler 54. As a result of processing in the separator 50, the contaminants 24 are removed from the ionic liquid and solvent. Specifically, a solvent, which may be water, is used to remove the contaminants 24 from the ionic liquid. A stream 56 of cleaned ionic liquid and solvent exits the separator 50 and is fed into an evaporator 58.

At the evaporator 58, the stream 56 of cleaned ionic liquid and solvent are separated into a regenerated ionic liquid stream 60 and a regenerated solvent stream 62. The regenerated ionic liquid stream 60 can be recycled into the extraction apparatus 36 to reduce the system need for ionic liquid. Likewise, the regenerated solvent stream 62 can be recycled into the extraction stage 44 to reduce the system demand for solvent.

Turning to the separation section 26, it can be seen that the clean stream of hydrocarbons 43 is delivered to an adsorbent chamber 64 via a rotary valve 66. A molecular sieve 68 such as a zeolite is contained in the adsorbent chamber 64. An exemplary zeolite would include a Linde Type A zeolite to extract normal paraffins, and silicalite to extract lightly branched paraffins. Desired hydrocarbons within the clean stream of hydrocarbons 43 (whether only normal paraffins or normal paraffins and lightly-branched paraffins) are more readily adsorbed by the molecular sieve 68 than non-desired hydrocarbons (whether non-normal paraffins or only heavier-branched paraffins). As a result, maintaining a contact zone between the clean stream of hydrocarbons 43 and the molecular sieve 68 over time results in the separation of desired hydrocarbons and non-desired hydrocarbons within the adsorbent chamber 64. To provide for extended contact, the adsorbent chamber 64 provides recirculating line 69 to circulate fluid from the bottom of the chamber to the top.

During this process of separation, desorbent 70 is introduced into the adsorbent chamber 64 to separate the hydrocarbons from the molecular sieve 68. In this process, the rotary valve 66 controls the input of the clean stream of hydrocarbons 43 and the desorbent 70 into the adsorbent chamber 64 as well as the removal of an extract 72 containing desorbent 70 and desired hydrocarbons and a raffinate 74 containing desorbent 70 and non-desired hydrocarbons from the chamber 64.

From the rotary valve, the extract 72 passes to an extraction column 76. In the extraction column 76, the desired hydrocarbons forming the desired paraffin product 14 are separated from the desorbent 70, which is recycled back to the adsorbent chamber 64.

Also from the rotary valve, the raffinate 74 passes to a raffinate column 78. In the raffinate column 78, the desorbent 70 is separated from the non-desired hydrocarbons 28 and returned to the adsorbent chamber 64. The non-desired hydrocarbons 28, whether all non-normals or only heavier-branched non-normals may be utilized for a number of other industrial purposes.

Accordingly, as may be understood from the discussion of the apparatus 10, a process is provided herein for producing a normal or substantially linear paraffin product from a kerosene feed including normal hydrocarbons, non-normal hydrocarbons and contaminants. In considering the hydrocarbon flow through the process, it can be seen that the kerosene feed is first fractionated to obtain a heart cut of kerosene containing hydrocarbons of desired molecular weights. Thereafter, the heart cut of kerosene is contacted with an ionic liquid stream to extract the contaminants into the ionic liquid stream to form a clean stream of hydrocarbons. If ionic liquid remains in the clean stream of hydrocarbons, the clean heart cut can be treated with a solvent stream in single stage or multiple stages to extract any ionic liquid. Mild hydroprocessing may then be used for further removal of contaminants.

Then, the clean stream of hydrocarbons is contacted with a molecular sieve. The sieve removes the desired hydrocarbons from the non-desired hydrocarbons in the clean stream of hydrocarbons. Thereafter, the desired hydrocarbons are recovered from the molecular sieve by a desorbent. Then, the desorbent is removed from the desired hydrocarbons, yielding the normal or substantially linear paraffin product.

In consideration of the flow of ionic fluid through the apparatus 10, it is seen that ionic fluid is contacted with the dirty heart cut of kerosene. As a result, contaminants such as heteroatoms containing nitrogen, sulfur, and oxygenates are extracted into the ionic fluid.

As a result of the processes and apparatuses for producing a normal or substantially linear paraffin product from a kerosene feed provided herein, the costs of desired paraffin production are significantly reduced. Specifically, the use of ionic liquids to pretreat of kerosene feed for contaminant removal renders hydrotreatment unnecessary, or enables the use of hydroprocessing at mild conditions. In either case, the process may be simplified through the avoidance of high pressure process equipment, hydrotreating catalysts, or sulfur recovery for converting hydrogen sulfide.

While at least one exemplary embodiment has been presented in the foregoing Detailed Description, it should be appreciated that a vast number of variations exist. It should also be appreciated that the exemplary embodiment or exemplary embodiments are only examples, and are not intended to limit the scope, applicability, or configuration of the invention in any way. Rather, the foregoing Detailed Description will provide those skilled in the art with a convenient road map for implementing an exemplary embodiment of the invention, it being understood that various changes may be made in the function and arrangement of elements described in an exemplary embodiment without departing from the scope of the invention as set forth in the appended Claims and their legal equivalents.

What is claimed is:

1. A process for producing a substantially linear paraffin product from a feed including normal hydrocarbons, lightly-branched non-normal hydrocarbons, heavier-branched non-normal hydrocarbons and contaminants, the process comprising:
contacting the feed with an ionic liquid stream and extracting the contaminants to form a clean stream of hydrocarbons;
selectively adsorbing the normal hydrocarbons and lightly-branched non-normal hydrocarbons from the clean stream of hydrocarbons with a molecular sieve to remove the normal hydrocarbons and lightly-branched non-normal hydrocarbons from the heavier-branched non-normal hydrocarbons;
recovering the normal hydrocarbons and lightly-branched non-normal hydrocarbons from the molecular sieve with a desorbent; and
separating the normal hydrocarbons and lightly-branched non-normal hydrocarbons from the desorbent to yield the substantially linear paraffin product.

2. The process of claim 1 wherein a portion of contaminants remain in the clean stream of hydrocarbons after contacting and extracting, the process further comprising processing the clean stream of hydrocarbons at mild hydroprocessing conditions to remove the remaining portion of contaminants therein.

3. The process of claim 1 wherein the feed is contacted with the ionic liquid stream in a counter-current extraction apparatus to form the clean stream of hydrocarbons.

4. The process of claim 1 further comprising:
before selectively adsorbing the normal hydrocarbons and lightly-branched non-normal hydrocarbons from the clean stream of hydrocarbons, treating the clean stream of hydrocarbons with a solvent stream to extract any ionic liquid from the clean stream of hydrocarbons.

5. The process of claim 1 wherein a dirty ionic liquid stream is formed by extracting the contaminants into the ionic liquid stream, the process further comprising:
mixing solvent with the dirty ionic liquid stream to remove the contaminants from the dirty ionic liquid and forming a cleaned ionic liquid stream.

6. The process of claim 5 further comprising:
removing solvent from the cleaned ionic liquid stream and forming a regenerated ionic liquid stream for recycle to the ionic liquid stream.

7. The process of claim 6 further comprising:
before selectively adsorbing the normal hydrocarbons and lightly-branched non-normal hydrocarbons from the clean stream of hydrocarbons, treating the clean stream of hydrocarbons with a solvent stream to extract any ionic liquid from the clean stream of hydrocarbons, wherein the solvent stream includes the solvent removed from the cleaned ionic liquid stream.

8. The process of claim 1 wherein the heavier-branched non-normal hydrocarbons include isoparaffins and aromatics, and further comprising yielding a raffinate stream of heavier-branched non-normal hydrocarbons after the step of selectively adsorbing.

9. The process of claim 1 further comprising:
fractionating a feedstock to obtain the feed, wherein the feed comprises C₅ to C₁₀ hydrocarbons.

10. The process of claim 1 further comprising:
fractionating a feedstock to obtain the feed, wherein the feed comprises C₁₀ to C₁₃ hydrocarbons.

11. The process of claim 1 further comprising:
fractionating a feedstock to obtain the feed, wherein the feed comprises C₁₀ to C₁₈ hydrocarbons.

12. A process for producing a normal paraffin product from a feed including normal hydrocarbons, non-normal hydrocarbons and contaminants, the process comprising:
contacting the feed with an ionic liquid stream and extracting contaminants from the feed into the ionic liquid stream to create a stream of clean stream of hydrocarbons and a stream of dirty ionic liquid;
feeding the stream of clean hydrocarbons to a molecular sieve, wherein the molecular sieve selectively adsorbs normal hydrocarbons from the clean stream of hydrocarbons;
recovering the normal hydrocarbons from the molecular sieve with a desorbent; and
separating the normal hydrocarbons from the desorbent to yield the normal paraffin product.

13. The process of claim 12 wherein a portion of contaminants remain in the clean stream of hydrocarbons after contacting and extracting, the process further comprising processing the clean stream of hydrocarbons at mild hydroprocessing conditions to remove the remaining portion of contaminants therein.

14. The process of claim 12 further comprising:
before feeding the clean stream of hydrocarbons to the molecular sieve, treating the clean stream of hydrocarbons with a solvent stream to extract any ionic liquid from the clean stream of hydrocarbons.

15. The process of claim 14 further comprising:
mixing the solvent stream with the dirty ionic liquid stream to remove the contaminants from the dirty ionic liquid to form a cleaned ionic liquid stream.

16. The process of claim 15 further comprising:
removing the solvent from the cleaned ionic liquid stream to form a regenerated ionic liquid stream for recycle to the ionic liquid stream and to form a regenerated solvent stream for recycle to the solvent stream.

17. The process of claim 12 further comprising:
fractionating a feedstock to obtain the feed, wherein the feed comprises C₅ to C₁₀ hydrocarbons.

18. The process of claim 12 further comprising:
fractionating a feedstock to obtain the feed, wherein the feed comprises C₁₀ to C₁₃ hydrocarbons.

19. The process of claim 12 further comprising:
fractionating a feedstock to obtain the feed, wherein the feed comprises C₁₀ to C₁₈ hydrocarbons.

20. An apparatus for producing a substantially linear paraffin product from a feed including normal hydrocarbons, lightly-branched non-normal hydrocarbons, heavier-branched non-normal hydrocarbons and contaminants, the apparatus comprising:
an extraction column configured to contact the feed with an ionic liquid stream to extract the contaminants from the feed into the ionic liquid stream to form a clean stream of hydrocarbons and a dirty ionic liquid stream
an adsorbent chamber configured to receive the clean stream of hydrocarbons;
a molecular sieve located in the adsorbent chamber configured to selectively adsorb the normal hydrocarbons and lightly-branched non-normal hydrocarbons from the clean stream of hydrocarbons to remove the normal hydrocarbons and lightly-branched non-normal hydrocarbons from the heavier-branched non-normal hydrocarbons to create a raffinate;
a desorbent configured to recover the normal hydrocarbons and lightly-branched non-normal hydrocarbons from the molecular sieve to create an extract;
an extract column configured to receive the extract and separate the normal hydrocarbons and lightly-branched non-normal hydrocarbons from the desorbent to yield the substantially linear paraffin product;
a raffinate column configured to receive the raffinate and separate the heavier-branched non-normal hydrocarbons from the desorbent;
an extraction stage configured to treat the clean stream of hydrocarbons with a solvent stream to extract any ionic liquid from the clean stream of hydrocarbons and to produce a solvent and ionic liquid stream;
a separator configured to receive the solvent and ionic liquid stream, to mix solvent with the dirty ionic liquid stream to remove the contaminants from the dirty ionic liquid, and to form a cleaned ionic liquid stream; and
an evaporator configured to remove the solvent from the cleaned ionic liquid stream to form a regenerated ionic liquid stream for recycling to the ionic liquid stream and a regenerated solvent stream for recycling to the extraction stage.
